(19) United States
(12) Patent Application Publication
(10) Pub. No.: US 2022/0125331 A1
(43) Pub. Date: Apr. 28, 2022

(54) SKIN CONDITION MEASUREMENT SENSOR MODULE AND SKIN CONDITION MEASUREMENT DEVICE

(71) Applicants: AMOSENSE CO., LTD., Cheonan-si, Chungcheongnam-do (KR); AMOREPACIFIC CORPORATION, Seoul (KR)

(72) Inventors: Byoung Su JIN, Cheonan-si (KR); Jeong Eun SEO, Seoul (KR); Sung Won YI, Seoul (KR); Jin NAM, Seoul (KR)

(73) Assignees: AMOSENSE CO., LTD., Cheonan-si, Chungcheongnam-do (KR); AMOREPACIFIC CORPORATION, Seoul (KR)

(21) Appl. No.: 17/429,974

(22) PCT Filed: Apr. 7, 2020
(86) PCT No.: PCT/KR2020/004661

§ 371 Date: Aug. 11, 2021

(30) Foreign Application Priority Data
Apr. 18, 2019 (KR) 10-2019-0045387

(51) Int. Cl. A61B 5/0537; A61B 5/00; C08J 5/18; A61B 5/01; H04W 4/80

(52) U.S. Cl. CPC A61B 5/0537; C08J 2379/08; A61B 5/443; C08J 5/18; A61B 5/002; A61B 5/01; C08J 2325/06; C08J 2327/18; A61B 2560/0214; A61B 2562/0215; A61B 2562/164; C08J 2323/08; A61B 2562/066; C08J 2327/16; C08J 2367/02; H04W 4/80; A61B 5/6898

(57) ABSTRACT

A skin condition measurement sensor module and a skin condition measurement device are provided. A skin condition measurement sensor module according to one embodiment contains: a transparent film; a pair of sensor electrodes disposed to be spaced at regular intervals at a plurality of positions on one surface of the transparent film; and a floating electrode formed on the entirety of the one surface of the transparent film, formed to be divided into predetermined sizes, and formed to be separated from the pair of sensor electrodes, wherein the sensor electrodes and the floating electrode are formed of a grid-shaped pattern layer.

### TECHNICAL FIELD

The present invention relates to a skin condition measurement sensor and a skin condition measurement device, and more specifically, to a skin condition measurement sensor module and a skin condition measurement device which satisfy both skin condition measurement and sensing sensitivity for touch recognition in a mobile communication terminal.

### BACKGROUND ART

Generally, since moisturizing is important to maintain skin health, it is important to maintain and manage skin moisture in daily life. Specifically, in the case of facial skin, much attention is focused on management. To this end, a device which measures a skin condition using a portable skin measurement device or a portable electronic device has been developed.

However, in order to measure the skin condition, there is the inconvenience of having a separate measurement device having a relatively large size. Further, a portable electronic device having a skin measurement sensor consumes extra time to measure the skin condition or requires management and attention for measurement and thus is cumbersome to use, and since a battery of the portable electronic device is consumed when measuring the skin condition, the use of the portable electronic device is restricted or use thereof is not possible when the remaining amount of the battery is small.

### DISCLOSURE

### Technical Problem

The present invention is directed to providing a skin condition measurement sensor module and a skin condition measurement device capable of satisfying both sensing sensitivity of measuring a user's skin condition with only a metal mesh and sensing sensitivity of recognizing a user's touch on a touchpad of a mobile communication terminal by enabling touch recognition.

Further, the present invention is directed to providing a skin condition measurement device capable of measuring a user's skin condition without using power of a mobile communication terminal by generating and providing power through energy harvesting during a call of the mobile communication terminal.

### Technical Solution

One aspect of the present invention provides a skin condition measurement sensor module including: a transparent film; a pair of sensor electrodes disposed to be spaced apart at regular intervals at a plurality of positions on one surface of the transparent film; and a floating electrode formed on the entirety of the one surface of the transparent film, formed to be divided into a predetermined size, and formed to be separated from the pair of sensor electrodes, wherein the sensor electrodes and the floating electrode are formed of a grid-shaped pattern layer.

According to a preferable embodiment of the present invention, the pair of sensor electrodes may be formed in a concavo-convex shape and may include a first sensor electrode and a second sensor electrode. In this case, the floating electrode may be disposed in concave portions of the first sensor electrode and the second sensor electrode.

Each of the pair of sensor electrodes may be formed in a comb shape, and a maximum width of each of the pair of sensor electrodes may be less than 1 to 20 mm.

Further, the transparent film may include one selected from polystyrene (PS), polyethylene terephthalate (PET), polybutylene terephthalate (PBT), polyethylene naphthalate (PEN), polyimide (PI), polytetrafluoroethylene (PTFE), a liquid crystal polymer (LCP), fluorinated ethylene propylene (PEP), perfluoroalkoxy (PFA), an ethylene-tetrafluoroethylene copolymer (ETFE), polyvinylidene fluoride (PVDF), an ethylene-chlorotrifluoroethylene copolymer (ECTFE), polychlorotrifluoroethylene (PCTFE), and a combination thereof.

In addition, the pattern layer may include one selected from aluminum (Al), titanium (Ti), chromium (Cr), iron (Fe), cobalt (Co), nickel (Ni), copper (Cu), zinc (Zn), ruthenium (Ru), palladium (Pd), silver (Ag), tin (Sn), neodymium (Nd), tungsten (W), platinum (Pt), gold (Au), molybdenum (Mo), stainless steel (SUS), and a combination thereof.

Alternatively, the pattern layer may include one selected from indium tin oxide (ITO), indium zinc oxide (IZO), indium zinc tin oxide (IZTO), indium aluminum zinc oxide (IAZO), indium gallium zinc oxide (IGZO), indium gallium tin oxide (IGTO), aluminum zinc oxide (AZO), antimony tin oxide (ATO), gallium zinc oxide (GZO), IrOₓ, RuOₓ, TiO₂, and a combination thereof.

In this case, the pattern layer may be formed by one process selected from metal-organic chemical vapor deposition (MOCVD), hydride vapor phase epitaxy (HVPE), thermal deposition, e-beam deposition, laser deposition, sputtering, and ion plating.

Another aspect of the present invention provides a skin condition measurement device including: a skin condition measurement sensor module; a power generator configured to harvest energy to generate power during a call of a mobile communication terminal; a measurement part configured to measure moisture and a surrounding temperature of a user's skin based on a signal detected by the skin condition measurement sensor module; a near field communication (NFC) communication part configured to transmit the measured information to the mobile communication terminal; and a controller configured to control the measurement of the moisture and the surrounding temperature of the skin, generation of power, and data transmission to the mobile communication terminal. Here, the skin condition measurement sensor module of various embodiments having the above-described structure and characteristics may be used as the skin condition measurement sensor module.

According to a preferable embodiment of the present invention, the NFC communication part may generate power by energy harvesting during communication

Further, the power generator may include: a rectenna configured to harvest energy during a call of the mobile communication terminal; a super capacitor configured to store the harvested energy; and a regulator configured to adjust the stored energy to a predetermined voltage.

In addition, the NFC communication part may include: a dynamic NFC tag configured to store the measured information and perform the energy harvesting during NFC communication; and an NFC antenna configured to transmit an NFC signal.

### Advantageous Effects

According to the present invention, by forming a floating electrode separated from a sensor electrode by a predetermined unit with a metal mesh, a skin condition is measured by the sensor electrode and a user's touch can be recognized by a touchpad of a mobile communication terminal by the floating electrode, and thus a skin condition measurement function using the mobile communication terminal can be generalized.

Further, in the present invention, since power is generated and provided through energy harvesting using a rectenna and a dynamic near field communication (NFC) tag, a user's skin condition can be measured without receiving power from the mobile communication terminal, and it is possible to use regardless of battery power of the mobile communication terminal, thereby improving convenience of use.

In addition, in the present invention, since the skin condition can be measured during a call, the user does not need to spend extra time to measure the skin condition, or there is no need for special management or attention for the measurement, and thus the hassle of skin condition measurement can be addressed.

### BRIEF DESCRIPTION OF DRAWINGS

FIG. 1 is a perspective view illustrating a state in which a skin condition measurement sensor module according to one embodiment of the present invention is attached to a mobile communication terminal.

FIG. 2 is an exploded perspective view illustrating a coupling state of a skin condition measurement device and the mobile communication terminal according to one embodiment of the present invention.

FIG. 3 is an enlarged view of one example of the skin condition measurement sensor module according to one embodiment of the present invention.

FIG. 4 is an enlarged view of another example of the skin condition measurement sensor module according to one embodiment of the present invention.

FIG. 5 is a side view illustrating a state in which the skin condition measurement device according to one embodiment of the present invention is coupled to the mobile communication terminal.

FIG. 6 is a perspective view illustrating a state in which the skin condition measurement sensor module according to one embodiment of the present invention is coupled to a case of the mobile communication terminal.

FIG. 7 is a disposition view of a circuit module of the skin condition measurement device according to one embodiment of the present invention, and

FIG. 8 is a block diagram of FIG. 7.

### MODES OF THE INVENTION

Hereinafter, exemplary embodiments of the present invention will be described in detail with reference to the accompanying drawings which may allow one of ordinary skill in the art to easily carry out the present invention. The present invention may be implemented in various forms and is not limited to the following embodiments. Components not related to the description are not included in the drawings to clearly describe the present invention, and the same reference symbols are used for the same or similar components in the description.

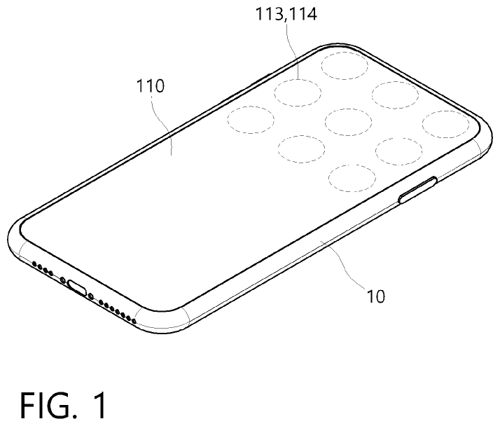

Referring to FIGS. 1 to 5, a skin condition measurement sensor module 110 according to one embodiment of the present invention includes a transparent film 111, a pair of sensor electrodes 113 and 114, and floating electrodes 116.

The skin condition measurement sensor module 110 may be attached to a touch screen of a mobile communication terminal 10. Here, the mobile communication terminal 10 may be a portable electronic device capable of making a call, such as a smartphone, and having a display part including a touch screen. In this case, the mobile communication terminal 10 may have a protective film attached thereto. That is, the skin condition measurement sensor module 110 may be attached on the touch screen of the mobile communication terminal 10 or on a protective film attached to the touch screen.

The transparent film 111 is made of a film to form a metal mesh. Further, the transparent film 111 is attached to the touch screen of the mobile communication terminal 10, and thus has a transparent material so that display of the mobile communication terminal 10 may be clearly seen.

For example, the transparent film 111 may be one selected from polystyrene (PS), polyethylene terephthalate (PET), polybutylene terephthalate (PBT), polyethylene naphthalate (PEN), polyimide (PI), polytetrafluoroethylene (PTFE), a liquid crystal polymer (LCP), fluorinated ethylene propylene (PEP), perfluoroalkoxy (PFA), an ethylene-tetrafluoroethylene copolymer (ETFE), polyvinylidene fluoride (PVDF), an ethylene-chlorotrifluoroethylene copolymer (ECTFE), polychlorotrifluoroethylene (PCTFE), and a combination thereof.

Here, since polyethylene terephthalate (PET) and polyimide (PI) have a property that has excellent insulation, heat resistance, and bending resistance, is flexible, has little dimensional deformation, and is strong against heat, polyethylene terephthalate (PET) or polyimide (PI) may be used.

In this case, a thickness of the transparent film 111 is different according to use and is not particularly limited, but may be 10 to 150 μm. Preferably, the thickness of the transparent film 111 may be 100 μm. Here, when the thickness of the transparent film 111 is less than 10 μm, it may be difficult to support or handle the sensor electrodes 113 and 114, and the possibility of damage by an external force may be high. On the contrary, when the thickness of the transparent film 111 exceeds 150 μm, flexibility is degraded.

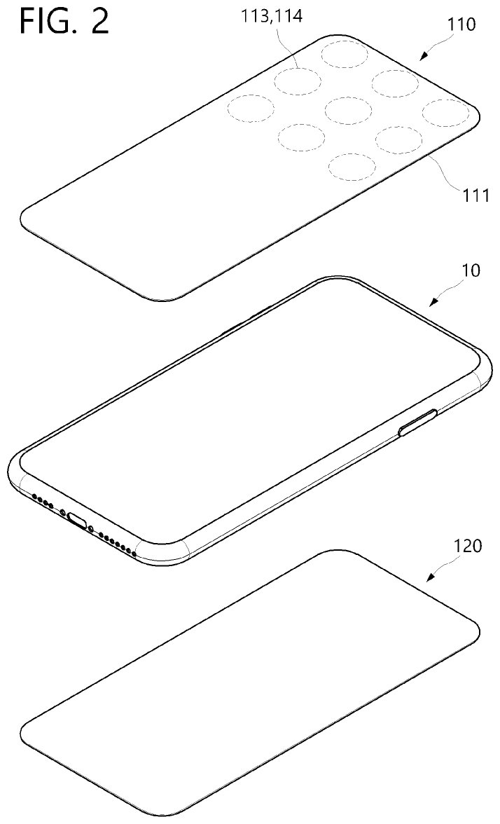

The pair of sensor electrodes 113 and 114 are formed at a plurality of positions on one surface of the transparent film 111. Referring to FIGS. 1 and 2, the sensor electrodes 113 and 114 may be formed at a plurality of positions on an upper end portion of the transparent film 111. Here, the upper end portion may be a position corresponding to a speaker of the mobile communication terminal 10. That is, when a user makes a call using the mobile communication terminal 10, the sensor electrodes 113 and 114 may be formed in a portion which comes into contact with a face of the user. For example, the sensor electrodes 113 and 114 may be disposed in nine positions in a 3×3 array, but the number is not particularly limited.

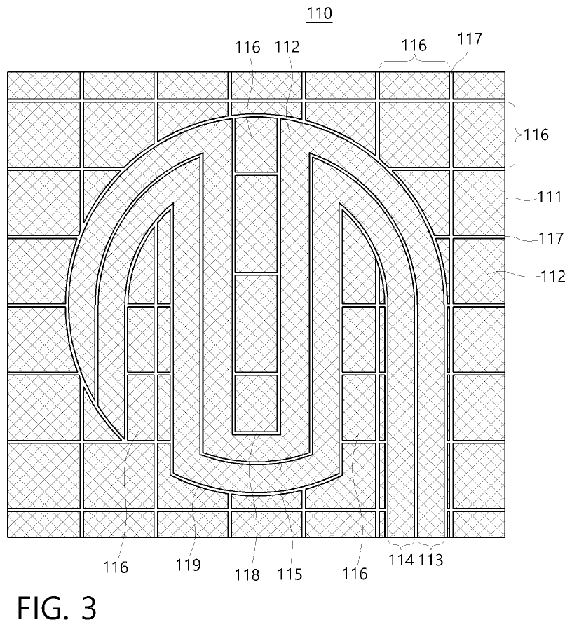

In this case, the sensor electrodes 113 and 114 are spaced apart from each other at a predetermined interval. Referring to FIG. 3, the sensor electrodes 113 and 114 may be formed with a first gap 115 interposed therebetween. That is, the sensor electrodes 113 and 114 may measure a capacitance through the first gap 115.

Further, the sensor electrodes 113 and 114 are generally formed in a concavo-convex shape. That is, in the sensor electrodes 113 and 114, concave portions and convex portions may be repeatedly formed. In this case, the sensor electrodes 113 and 114 may be disposed in parallel.

Here, the sensor electrodes 113 and 114 are formed of a grid-shaped pattern layer 112. That is, the sensor electrodes 113 and 114 may be formed of a metal mesh.

Further, the sensor electrodes 113 and 114 may include a first sensor electrode 113 and a second sensor electrode 114. In this case, the floating electrodes 116, which will be described below, may be respectively formed in the concave portions of the first sensor electrode 113 and the second sensor electrode 114.

Accordingly, even when the user touches near the sensor electrodes 113 and 114, the user's touch may be recognized by the floating electrodes 116. That is, when the sensor electrodes 113 and 114 are densely formed in a comb shape, the user's touch is not recognized. Accordingly, in order to form a space between the sensor electrodes 113 and 114, the sensor electrodes 113 and 114 may be formed in the concavo-convex shape, and the floating electrodes 116 may be formed in the concave portions which are those spaces.

Accordingly, since the skin condition may be measured by the sensor electrodes 113 and 114 and the user's touch may be recognized by the floating electrodes 116, the skin condition measurement sensor module 110 may be attached on the touch screen of the mobile communication terminal 10. Accordingly, the skin condition measurement sensor module 110 may be used in any mobile communication terminal 10, and thus applicability of the skin condition measurement sensor module 110 may be improved, and a skin condition measurement function using the mobile communication terminal 10 may be generalized.

The floating electrode 116 is formed on an entire surface of one surface of the film. In this case, the floating electrodes 116 are divided into predetermined sizes. That is, the floating electrodes 116 may be formed with a second gap 117 interposed therebetween.

Accordingly, the user's touch may be recognized in a portion that the user touches by the floating electrodes 116. That is, the user's touch may be directly transmitted to the touch screen of the mobile communication terminal 10 disposed under the floating electrodes 116.

In this case, when the floating electrodes 116 are formed only on a part of the transparent film 111, the visibility of the skin condition measurement sensor module 110 may vary according to an angle. That is, a portion where the floating electrode 116 is formed and a portion where the floating electrode 116 is not formed may be distinguished as a stain. Accordingly, the floating electrodes 116 may be provided throughout the transparent film 111 so that the user may have the same visibility over the entire skin condition measurement sensor module 110.

Further, the floating electrodes 116 are formed to be separated from the sensor electrodes 113 and 114 through a third gap 118 and a fourth gap 119. That is, the first sensor electrode 113 may be separated from the floating electrode 116 through the third gap 118, and the second sensor electrode 114 may be separated from the floating electrode 116 through the fourth gap 119.

Here, the floating electrode 116 is formed of the grid-shaped pattern layer 112. That is, the floating electrode 116 may be formed of a metal mesh.

In this case, the sensor electrodes 113 and 114 and the pattern layer 112 of the floating electrodes 116 may be one selected from aluminum (Al), titanium (Ti), chromium (Cr), iron (Fe), cobalt (Co), nickel (Ni), copper (Cu), zinc (Zn), ruthenium (Ru), palladium (Pd), silver (Ag), tin (Sn), neodymium (Nd), tungsten (W), platinum (Pt), gold (Au), molybdenum (Mo), stainless steel (SUS), and a combination thereof

Alternatively, the pattern layer 112 may be a transparent electrode. For example, the pattern layer 112 may be one selected from indium tin oxide (ITO), indium zinc oxide (IZO), indium zinc tin oxide (IZTO), indium aluminum zinc oxide (IAZO), indium gallium zinc oxide (IGZO), indium gallium tin oxide (IGTO), aluminum zinc oxide (AZO), antimony tin oxide (ATO), gallium zinc oxide (GZO), IrOₓ, RuOₓ, TiO₂, and a combination thereof.

Further, the pattern layer 112 may have a thickness of 0.01 to 2 μm. Preferably, the pattern layer 112 may have a thickness of 1 μm. When the thickness of the pattern layer 112 is less than 0.01 μm, there are difficulties in a manufacturing process, and when the thickness of the pattern layer 112 exceeds 2 μm, the thickness of the skin condition measurement sensor module 110 becomes thick and the pattern layer 112 collapses.

Here, the pattern layer 112 may form a grid with a line width of 1 to 5 μm and an interval of 150 to 400 μm. Here, when the line width is less than 1 μm, sensitivity for the skin condition measurement and the user's touch is reduced, and when the line width exceeds 5 μm, the visibility on a front surface of the transparent film 111 is reduced. Further, when the interval of the pattern layer 112 is less than 150 μm, sensitivity for the skin condition measurement and the user's touch is reduced, and when the interval of the pattern layer 112 exceeds 400 μm, stains or irregular patterns may be seen on the front of the transparent film 111.

In this case, the second gap 117 forming the floating electrode 116 may be formed at an interval of 50 to 200 μm. That is, the floating electrodes 116 may be separated by an interval of 50 μm to 200 μm. Preferably, the floating electrodes 116 may be separated by an interval of 100 μm.

When the interval between the floating electrodes 116 is less than 50 μm, an error may occur due to a reduction in the sensitivity for the user's touch, and when the interval between the floating electrodes 116 exceeds 200 μm, stains or irregular patterns may be seen on the front surface of the transparent film 111 due to the pattern layer 112.

Such a pattern layer 112 may be formed using a vacuum deposition method. Here, the vacuum deposition method includes chemical vapor deposition (CVD) using a chemical method and physical vapor deposition (PVD) using a physical method. The CVD includes metal-organic chemical vapor deposition (MOCVD) and hydride vapor phase epitaxy (HVPE), and the like. The PVD includes thermal evaporation, e-beam deposition, laser deposition, sputtering, ion plating, and the like. Preferably, the pattern layer 112 may be formed by a sputtering process.

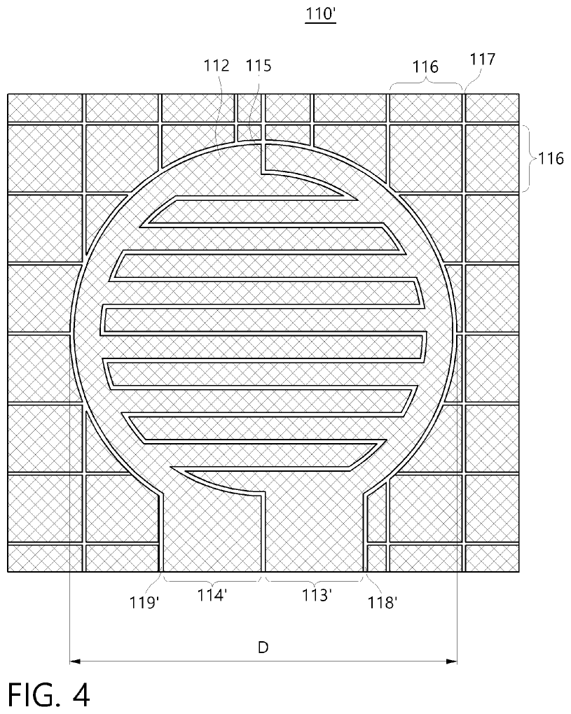

Alternatively, the skin condition measurement sensor module may include a comb-shaped sensor electrode. Referring to FIG. 4, in a skin condition measurement sensor module 110′, each of sensor electrodes 113′ and 114′ may be formed in a comb shape. Here, the sensor electrodes 113′ and 114′ may be disposed to be engaged with each other with a first gap 115 interposed therebetween.

Further, the sensor electrodes 113′ and 114′ may each have a circular shape as a whole. That is, the sensor electrodes 113′ and 114′ are separated from floating electrodes 116 with a third gap 118′ or a fourth gap 119′ interposed therebetween. In this case, the third gap 118′ and the fourth gap 119′ forming an outer appearance of the sensor electrodes 113′ and 114′ may each have a substantially circular shape. However, the present invention is not limited thereto, and the sensor electrodes 113′ and 114′ may be formed in various shapes such as a quadrangular shape and the like as a whole.

Further, the sensor electrodes 113′ and 114′ have a maximum width, for example, a diameter D of a circle formed by the third gap 118′ and the fourth gap 119′ in FIG. 4 may be 1 to 20 mm. Here, when the diameter D is less than 1 mm, sensitivity for detecting the skin condition by the sensor electrodes 113′ and 114′ is not satisfied, and when the diameter D exceeds 20 mm, an error may occur due to a high degree of interference with the user's touch.

Further, since the skin condition measurement sensor module 110′ is the same as the skin condition measurement sensor module 110 illustrated in FIG. 3 except for the shapes of the above-described sensor electrodes 113′ and 114′, specific descriptions will be omitted.

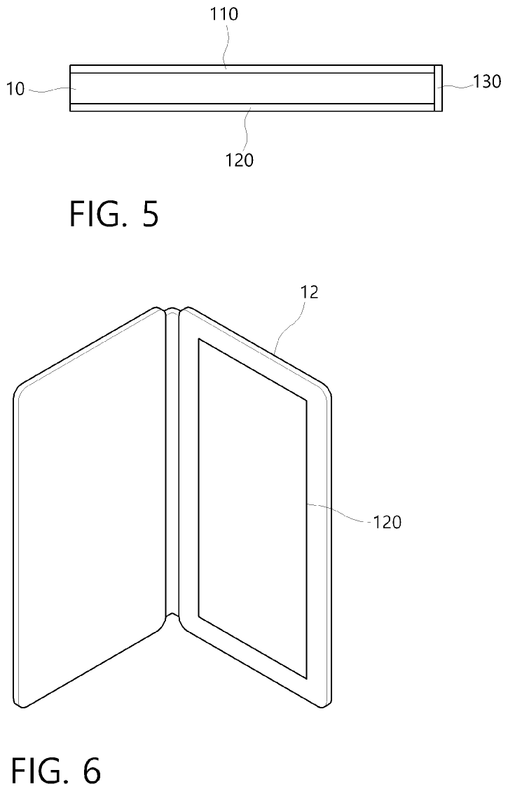

Referring to FIGS. 2 and 5, a skin condition measurement device 100 according to the embodiment of the present invention includes the skin condition measurement sensor module 110, a circuit module 120, and a connection part 130.

The skin condition measurement sensor module 110 may be disposed on a front surface (that is, the touch screen) of the mobile communication terminal 10 and the circuit module 120 may be disposed on a back surface (that is, a back cover) of the mobile communication terminal 10. In this case, the skin condition measurement sensor module 110 and the circuit module 120 may be connected through the connection part 130.

The connection part 130 may be a flexible connection terminal. For example, the connection part 130 may be a flexible connection terminal which is bent so as to connect the skin condition measurement sensor module 110 and the circuit module 120. In this case, in the connection part 130, it is possible to increase the number of terminals in an array manner to uniformly come into contact with terminals connected to the skin condition measurement sensor module 110 and terminals connected to the circuit module 120 upon contact.

Referring to FIG. 6, the circuit module 120 may be disposed in an external case 12 of the mobile communication terminal. That is, the circuit module 120 is disposed on the back surface of the mobile communication terminal 10, and when the mobile communication terminal 10 is mounted on the external case 12, the circuit module 120 may be attached to a position where the mobile communication terminal 10 is mounted in the external case 12. In this case, after the circuit module 120 is attached to the external case 12 and the mobile communication terminal 10 whose front surface is attached to the skin condition measurement sensor module 110 is mounted in the case, the skin condition measurement sensor module 110 and the circuit module 120 may be connected through the connection part 130.

Here, the external case 12 is illustrated and described with a wallet-type case as an example, but is not limited thereto, and may include a silicone case which covers only the back surface of the mobile communication terminal 10.

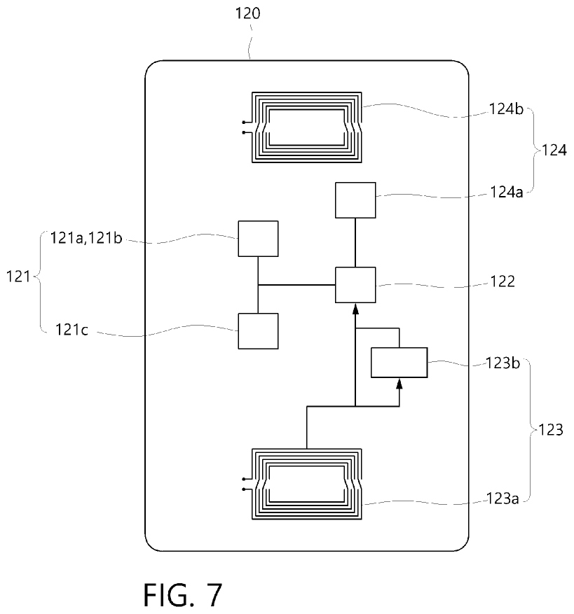

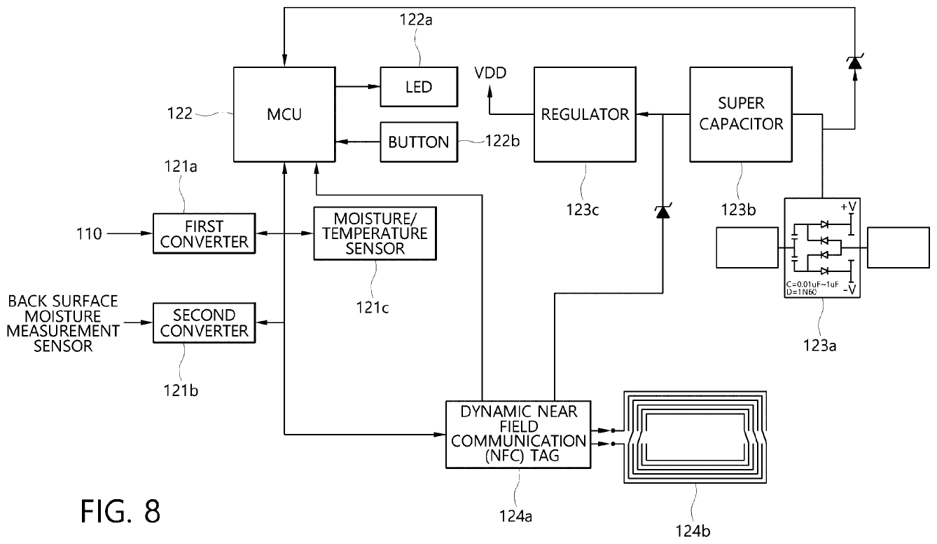

Referring to FIGS. 7 and 8, the circuit module 120 includes a measurement part 121, a micro controller unit (MCU) 122, a power generator 123, and a near field communication (NFC) communication part 124.

The measurement part 121 measures moisture and a surrounding temperature of the user's skin based on a signal detected by the skin condition measurement sensor module 110. Further, the measurement part 121 may be disposed in a portion adjacent to the skin condition measurement sensor module 110 and the connection part 130, and may include converters 121a and 121b and a moisture/temperature sensor 121c.

A first converter 121a may convert the capacitance measured by the skin condition measurement sensor module 110 disposed on the front surface of the mobile communication terminal 10 to a digital value. Here, it is possible to select and convert the most stable value among the plurality of sensor electrodes 113 and 114 of the skin condition measurement sensor module 110.

A second converter 121b may convert the capacitance measured by a moisture measurement sensor module disposed on the back surface of the mobile communication terminal 10 into a digital value. Here, the moisture measurement sensor module disposed on the back surface of the mobile communication terminal 10 is provided to measure skin moisture of a hand as an indirect reference when the user holds the mobile communication terminal 10.

In this case, it is possible to check whether there is skin-specific data within a reference value range by setting a lower portion of the skin measurement sensor held by a hand of the user during a call as a reference and setting an upper portion as a humidity sensing part which senses a relative measurement value of a reference.

The moisture/temperature sensor 121c may measure a moisture condition of the skin by the first converter 121a and the second converter 121b. Further, the moisture/temperature sensor 121c may measure the surrounding temperature.

The MCU 122 is disposed approximately in the center of the circuit module 120, and controls the measurement part 121, the power generator 123, and the NFC communication part 124. That is, the MCU 122 controls the measurement of skin moisture and the surrounding temperature, power generation, and data transmission to the mobile communication terminal 10. Further, the MCU 122 may be a low-power MCU to receive power by the energy harvesting of the power generator 123.

In this case, the MCU 122 may include a light emitting diode (LED) indicator 122a and an input button 122b (see FIG. 8). The LED indicator 122a may indicate that the skin condition is being measured by the skin condition measurement sensor module 110. The input button 122b may allow the user to select start or end of the measurement of the skin condition.

Alternatively, the MCU 122 may control the skin condition measurement to be automatically performed while the user makes a call through the mobile communication terminal 10. That is, the MCU 122 may measure the skin condition through the skin condition measurement sensor module 110 when the mobile communication terminal 10 is in a call regardless of an input of the input button 122b.

Accordingly, there is no need for the user to spend extra time for the skin condition measurement or to operate a button for the measurement, and there is no need for the user to take special care or attention for the measurement. Accordingly, since inconvenience of the condition measurement may be addressed, user convenience may be improved.

The power generator 123 generates power by harvesting energy during a call of the mobile communication terminal 10. Further, the power generator 123 may be disposed at a lower end of the circuit module 120 and may include a rectenna 123a, a super capacitor 123b, and a regulator 123c.

The rectenna 123a may harvest energy by collecting the power generated during a call of the mobile communication terminal 10. The rectenna 123a is a rectifying antenna, and may absorb radio waves during a call from the mobile communication terminal 10 and directly convert the radio waves to direct current power. Further, the rectenna 123a may absorb radio waves during communication of the mobile communication terminal 10 to convert direct current to power.

The super capacitor 123b may store energy harvested by the rectenna 123a. The regulator 123c supplies power VDD by adjusting the energy stored in the super capacitor 123b to a predetermined voltage. That is, the regulator 123c supplies the power VDD to the MCU 122, the LED indicator 122a, the input button 122b, the moisture/temperature sensor 121c, the first converter 121a, and the second converter 121b. Here, the super capacitor 123b and the regulator 123c may function as a power stabilization circuit.

Accordingly, it is possible to measure the skin condition by generating power by itself without the need to receive power from the mobile communication terminal 10. Accordingly, the power may be used regardless of the battery power of the mobile communication terminal 10, and thus convenience of use may be improved. That is, even when the remaining amount of the battery of the mobile communication terminal 10 is insufficient, use is not restricted, and since the battery is not consumed for skin measurement, a battery usage time is not reduced.

The NFC communication part 124 transmits information measured by the moisture/temperature sensor 121c to the mobile communication terminal 10. Further, the NFC communication part 124 is disposed at an upper end of the circuit module 120 and may include a dynamic NFC tag 124a and an NFC antenna 124b.

The dynamic NFC tag 124a may store the information measured by the moisture/temperature sensor 121c and transmit the information to the mobile communication terminal 10. In this case, the dynamic NFC tag 124a may perform harvesting. That is, the dynamic NFC tag 124a may absorb the radio waves from the NFC antenna 124b and convert the radio waves to DC power when the stored measurement information is transmitted to the mobile communication terminal 10.

In this case, the dynamic NFC tag 124a may transmit the harvested power to the regulator 123c. Accordingly, in addition to the energy harvested during the call of the mobile communication terminal 10, the energy harvested during NFC communication may be summed to supply the power VDD.

The NFC antenna 124b may transmit an NFC signal from the dynamic NFC tag 124a. Further, the NFC antenna 124b may exchange data with the mobile communication terminal 10.

In this case, the NFC communication part 124 may start communication after a delay for a predetermined time to avoid a collision with an NFC function of the mobile communication terminal 10 itself.

Although embodiments of the present invention have been described above, the spirit of the present invention is not limited to the embodiments shown in the description, and although those skilled in the art may provide other embodiments through the addition, change, or removal of the components within the scope of the same spirit of the present invention, such embodiments are also included in the scope of the spirit of the present invention.

1. A skin condition measurement sensor module comprising:
a transparent film;
a pair of sensor electrodes disposed to be spaced apart at regular intervals at a plurality of positions on one surface of the transparent film; and
a floating electrode formed on the entirety of the one surface of the transparent film, formed to be divided into a predetermined size, and formed to be separated from the pair of sensor electrodes,
wherein the sensor electrodes and the floating electrode are formed of a grid-shaped pattern layer.

2. The skin condition measurement sensor module of claim 1, wherein:
the pair of sensor electrodes are formed in a concavo-convex shape and comprises a first sensor electrode and a second sensor electrode; and
the floating electrode is disposed in concave portions of the first sensor electrode and the second sensor electrode.

3. The skin condition measurement sensor module of claim 1, wherein:
each of the pair of sensor electrodes is formed in a comb shape; and
a maximum width of each of the pair of sensor electrodes is less than 1 to 20 mm.

4. The skin condition measurement sensor module of claim 1, wherein the transparent film comprises one selected from polystyrene (PS), polyethylene terephthalate (PET), polybutylene terephthalate (PBT), polyethylene naphthalate (PEN), polyimide (PI), polytetrafluoroethylene (PTFE), a liquid crystal polymer (LCP), fluorinated ethylene propylene (PEP), perfluoroalkoxy (PFA), an ethylene-tetrafluoroethylene copolymer (ETFE), polyvinylidene fluoride (PVDF), an ethylene-chlorotrifluoroethylene copolymer (ECTFE), polychlorotrifluoroethylene (PCTFE), and a combination thereof.

5. The skin condition measurement sensor module of claim 1, wherein the pattern layer comprises one selected from aluminum (Al), titanium (Ti), chromium (Cr), iron (Fe), cobalt (Co), nickel (Ni), copper (Cu), zinc (Zn), ruthenium (Ru), palladium (Pd), silver (Ag), tin (Sn), neodymium (Nd), tungsten (W), platinum (Pt), gold (Au), molybdenum (Mo), stainless steel (SUS), and a combination thereof.

6. The skin condition measurement sensor module of claim 1, wherein the pattern layer comprises one selected from indium tin oxide (ITO), indium zinc oxide (IZO), indium zinc tin oxide (IZTO), indium aluminum zinc oxide (IAZO), indium gallium zinc oxide (IGZO), indium gallium tin oxide (IGTO), aluminum zinc oxide (AZO), antimony tin oxide (ATO), gallium zinc oxide (GZO), IrOₓ, RuOₓ, TiO₂, and a combination thereof.

7. The skin condition measurement sensor module of claim 1, wherein the pattern layer is formed by one process selected from metal-organic chemical vapor deposition (MOCVD), hydride vapor phase epitaxy (HVPE), thermal deposition, e-beam deposition, laser deposition, sputtering, and ion plating.

8. A skin condition measurement device comprising:
a skin condition measurement sensor module comprising a transparent film, a pair of sensor electrodes disposed to be spaced apart at regular intervals at a plurality of positions on one surface of the transparent film, and a floating electrode formed on the entirety of the one surface of the transparent film, formed to be divided into a predetermined size, and formed to be separated from the pair of sensor electrodes, wherein the sensor electrodes and the floating electrode are formed of a grid-shaped pattern layer;
a power generator configured to harvest energy to generate power during a call of a mobile communication terminal;
a measurement part configured to measure moisture and a surrounding temperature of a user's skin based on a signal detected by the skin condition measurement sensor module;
a near field communication (NFC) communication part configured to transmit the measured information to the mobile communication terminal; and
a controller configured to control the measurement of the moisture and the surrounding temperature of the skin, generation of power, and data transmission to the mobile communication terminal.

9. The skin condition measurement device of claim 8, wherein the NFC communication part generates power by energy harvesting during communication.

10. The skin condition measurement device of claim 8, wherein the power generator comprises a rectenna configured to harvest energy during a call of the mobile communication terminal;
a super capacitor configured to store the harvested energy; and
a regulator configured to adjust the stored energy to a predetermined voltage.

11. The skin condition measurement device of claim 9, wherein the NFC communication part comprises:
a dynamic NFC tag configured to store the measured information and perform the energy harvesting during NFC communication; and
an NFC antenna configured to transmit an NFC signal.
